(19) United States
(12) Patent Application Publication
(10) Pub. No.: US 2010/0041620 A1
(43) Pub. Date: Feb. 18, 2010

(54) METHODS FOR IMPROVING FRONTAL BRAIN BIOENERGETIC METABOLISM

(76) Inventors: Perry Renshaw, Salt Lake City, UT (US); Deborah Yurgelun-Todd, Salt Lake City, UT (US)

Correspondence Address: CLARK & ELBING LLP, 101 FEDERAL STREET, BOSTON, MA, 02110, US

(21) Appl. No.: 12/541,452

(22) Filed: Aug. 14, 2009

Related U.S. Application Data
(60) Provisional application No. 61/089,324, filed on Aug. 15, 2008.

(51) Int. Cl. A61K 31/7072; A61K 31/7068; A61P 25/00

(52) U.S. Cl. 514/50; 514/49

(57) ABSTRACT

The invention provides methods and compositions for augmenting bioenergetic metabolism in the frontal brain involving administration of a cytidine-containing or uridine-containing compound to a human.

### CROSS-REFERENCE TO RELATED APPLICATIONS

This application claims benefit of U.S. Provisional Application No. 61/089,324, filed Aug. 15, 2008, which is hereby incorporated by reference.

### BACKGROUND OF THE INVENTION

In general, the invention relates to the field of brain health.

Deficits in bioenergetic metabolism in the brain have been linked to harmful conditions, including loss of consciousness, cognitive impairment, psychiatric disorders, and heightened intolerance to oxygen deprivation. In addition, neuronal energy compromise may accelerate oxidative stress, which in turn, may contribute to aging-related memory loss and cognitive impairment. Thus, adequate energy stores serve a neuroprotective function.

There are to date no prescribed methods for prophylactic improvement of bioenergy stores in the brain of healthy individuals. Because these stores may prevent or mitigate future pathologies and promote optimal cognitive function, it would be desirable for healthy individuals to improve bioenergetic metabolism in the brain.

### SUMMARY OF THE INVENTION

In one aspect, the invention features a method of augmenting bioenergetic metabolism in the frontal brain of a human by administering a cytidine-containing or uridine-containing compound to the human, e.g., a neuropsychologically healthy adult or child.

In a preferred embodiment, the cytidine-containing or uridine-containing compound is CDP-choline, uridine, or triacetyl uridine; and the cytidine-containing or uridine-containing compound is provided in a nutraceutical composition, e.g., a drink or tablet, e.g., additionally containing vitamin B6, vitamin B12, niacin, folic acid, tyrosine, phenylalanine, taurine, malic acid, glucuronolactone, and/or caffeine; and the cytidine-containing or uridine-containing compound is administered orally.

In another embodiment, the cytidine-containing or uridine-containing compound is administered chronically, e.g., for a period of 3, 5, 10, 90, or 180 days or 1, 5, or 10 years.

In certain embodiments, the method may involve diagnosing a human as neuropsychologically healthy prior to administration of the cytidine-containing or uridine-containing compound.

In another aspect, the invention features a nutraccutical composition comprising an effective amount of a cytidine-containing or uridine-containing compound and optionally additional ingredients, e.g., vitamin B6, vitamin B12, niacin, folic acid, tyrosine, phenylalanine, taurine, malic acid, glucuronolactone, and/or caffeine, for administration to a neuropsychologically healthy human to augment bioenergetic metabolism in the frontal brain of the human.

In preferred embodiments of the invention, the cytidine-containing compound is cytidine, CDP, or CDP-choline; the cytidine-containing compound includes choline; and the human is a neuropsychologically healthy child or adult under the age of 60. In other preferred embodiments, the uridine-containing compound is uridine or triacetyl uridine.

In certain embodiments, the human does not have a neurological, psychiatric, or cognitive disorder, including, e.g., mood disorders (e.g., unipolar depression, dysthymia, cyclothymia, and bipolar disorder), attention-deficit hyperactivity disorder (ADHD), anxiety disorders (e.g., panic disorder and generalized anxiety disorder), obsessive-compulsive disorder (OCD), post-traumatic stress disorder (PTSD), phobias, and psychotic disorders (e.g., schizophrenia and schizoaffective disorder); does not have a sleep disorder (e.g., insomnia, constructive or obstructive sleep apnea, restless leg syndrome, periodic limb movements, problem sleepiness, and narcolepsy); has a normal sleep-wake cycle; has not had a stroke or other traumatic injury to the brain; is not using, abusing, withdrawing from, or dependent on a controlled substance, e.g., alcohol, stimulants (e.g., amphetamines, methamphetamine, methylphenidate, and cocaine), marihuana, and opiate or opioid drugs; does not use or is not dependent on tobacco or nicotine; or does not suffer from cardiovascular disease, cancer, dysmenorrhea, infertility, preeclampsia, postpartum depression, menopausal discomfort, osteoporosis, thrombosis, inflammation, hyperlipidemia, hypertension, rheumatoid arthritis, hyperglyceridemia, or gestational diabetes.

In other embodiments, the human is less than 30, 40, 50, or 60 years old.

By “neuropsychologically healthy human” is meant a person who does not have and has not been diagnosed with a neurological, cognitive, psychiatric, or sleep disorder, e.g., one listed herein; who is not suffering from sleep deprivation, disrupted sleep-wake cycles, head trauma, cerebral vasoconstriction sequelae, stroke, or other ischemic event in the brain; who does not have age-related dementia or cognitive decline; who is not using, abusing, withdrawing from, or dependent on a controlled substance, e.g., alcohol, stimulants including amphetamines, methamphetamine, methylphenidate, and cocaine, marihuana, and opiate or opioid drugs; and who is not using or dependent on tobacco or nicotine.

By “frontal brain” is meant the prefrontal cortex of the brain.

By anterior cingulate cortex” (ACC) is meant the structure within the prefrontal cortex that lies anterior to the genu of the corpus callosum.

By “parieto-occipital cortex” (POC) is meant the region of the parietal and occipital cortex thought to be involved in integration of sensory information, particularly visuospatial and visuomotor activities.

By “an effective amount” is meant an amount of a compound sufficient to augment bioenergetic metabolism in the brain.

By “augmenting bioenergetic metabolism” is meant increasing the amount or metabolism of energy stores. By “energy stores” is meant high-energy phosphate molecules that are metabolized to generate free energy. Examples of high-energy phosphate molecules include phosphocreatine and β-nucleoside triphosphates, e.g., adenosine triphosphate (ATP). Augmented bioenergetic metabolism may be evidenced by, e.g., an increase in glucose utilization or an increase in phosphocreatine, β-nucleoside triphosphates, or the ratio of phosphocreatine to inorganic phosphate. Augmented bioenergetic metabolism may be detected by, e.g., magnetic resonance spectroscopy imaging. The increase may be, e.g., a 2%, 5%, 10%, or 50% increase in the levels of energy stores or in the rate of metabolism in a subject, as compared to the levels and rate of metabolism in the subject prior to treatment with a cytidine-containing or uridine-containing compound. By “augmenting bioenergetic metabolism in the frontal brain” is meant increasing the amount or metabolism of energy stores in a part of the frontal brain, e.g., the ACC, in several parts of the frontal brain, e.g., the ACC, the orbitofrontal cortex, the doroslateral prefrontal cortex, or in the entire frontal brain, relative to the amount or metabolism of energy stores in a region outside of the frontal brain, e.g., the POC.

By “nutraceutical composition” is meant a composition having ingredients suitable at least for human consumption. Pharmaceutical grade ingredients may optionally be employed, as described, e.g., in “Remington: The Science and Practice of Pharmacy” (21st ed.) ed. A. R. Gennaro, 2005, Lippincott, Philadelphia, Pa.

By “cytidine-containing compound” is meant any compound that formally includes, as a component, cytidine, CMP, CDP, CTP, dCMP, dCDP, or dCTP. A compound is cytidine-containing if one or more hydrogen atoms of cytidine are replaced with another moiety.

By “uridine-containing compound” is meant any compound that formally includes, as a component, uridine, UMP, UDP, UTP, dUMP, dUDP, or dUTP. A compound is uridine-containing if one or more hydrogen atoms of uridine are replaced with another moiety. Uridine-containing compounds can include analogs of uridine, e.g., triacetyl uridine.

By “phospholipid” is meant a lipid containing phosphorus, e.g., phosphatidic acids (e.g., lecithin), phosphoglycerides, sphingomyelin, and plasmalogens. By “phospholipid precursor” is meant a substance that is incorporated into a phospholipid during synthesis of the phospholipid, e.g., fatty acids, glycerol, sphingosine, choline, and inositol.

By “child” is meant an individual who has not attained complete growth and maturity. Generally, a human child is under twenty-one years of age.

By “chronic” is meant over a period of longer than 2 days, e.g., over a period of 5, 10, or 90 days, or 1, 5, or 10 years.

The cytidine-containing and uridine-containing compounds utilized herein are relatively non-toxic, and CDP-choline, uridine, and triacetyl uridine, in particular, are pharmacokinetically understood and well tolerated by mammals. The present invention, therefore, provides methods and compositions that are likely to have few adverse effects and may be administered to children as well as mature adults.

Other features and advantages will be apparent from the following description and the claims.

### BRIEF DESCRIPTION OF THE DRAWINGS

FIG. 1 depicts localizer images used to confirm patient orientation and angle at baseline and six-week follow-up visits.

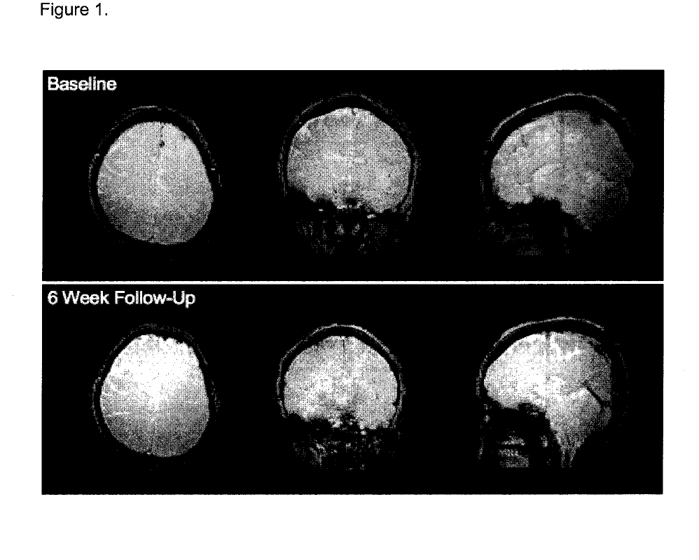

FIG. 2 depicts a three-dimensional magnetic resonance spectroscopy imaging (MRSI) grid used to image the anterior cingulate cortex (ACC) and parieto-occipital cortex (POC) of a subject. The 3D MRSI grid was shifted in the z dimension, using the sagittal image, to align the grid with the top of the corpus callosum. The portion of the MRSI grid encompassing the genu and splenium of the corpus callosum (left, sagittal image, outer green box) was then shifted in the x and y dimensions in the axial plane to align the MRSI grid (right, axial image, outer green box) with the longitudinal fissure. Two 25 cm³ voxels (effective size) were placed in each of the two regions of interest, the anterior cingulate cortex (ACC) and parietal/occipital cortex (POC) (right, axial image, small green boxes).

FIG. 3 depicts in vivo ³¹P spectra from the ACC and POC of a subject. Sample in vivo ³¹P brain spectra from 25 cm³ effective voxels in the ACC and the POC of a study participant at 4 Tesla. Raw data are displayed with the modeled fit and residual; 15 Hz exponential filtering has been applied for display. PME, phosphomonoester; PE, phosphoethanolamine; PC, phosphocholine; Pi, inorganic phosphate; PDE, phosphodiester; GPE, glycerophosphoethanolamine; GPC, glycerolphosphocholine; PCr, phosphocreatine; γ-NTP, γ-nucleoside triphosphate; α-NTP, α-nucleoside triphosphate; β-NTP, β-nucleoside triphosphate; ppm, parts per million.

### DETAILED DESCRIPTION OF THE INVENTION

In general, the invention features methods and compositions employing a cytidine-containing or uridine-containing compound to augment bioenergetic metabolism in the frontal brain of a human, e.g., a neuropsychologically healthy adult or child. An exemplary compound is CDP-choline.

Citicoline (CDP-choline; cytidine 5′-diphosphocholine) is a nucleotide that plays an important role in cellular metabolism, provides a source of membrane phospholipid precursors, serves as a catalyst for acetylcholine (ACh) synthesis, and modulates catecholaminergic neurotransmission. Citicoline also has been shown to reduce memory impairments associated with aging. Age-related declines in cognitive abilities, particularly related to frontal brain function, are thought to be due in part to decrements in oxygen and glucose consumption and reductions in cerebral blood flow (CBF). Additionally, a mitochondrial theory of aging has been suggested, which asserts that the aging process involves impairment of mitochondrial membrane proteins, declines in electron transport chain activity, and increases in oxidative stress resulting from mitochondrial respiratory metabolism.

Orally or intravenously administered citicoline is metabolized to choline and cytidine in the gastrointestinal system of the rat, with cytidine being further metabolized to uridine in the gastrointestinal system and liver of humans. Circulating uridine enters the brain via the blood brain barrier and undergoes phosphorylation to become uridine triphosphate (UTP), which is then converted to CTP (cytidine triphosphate) by CTP synthetase. Free choline undergoes phosphorylation to become phosphocholine, which in combination with CTP yields CDP-choline. Endogenous CDP-choline then reacts with diacylglycerol (DAG) to form phosphatidylcholine (PtdCho). Thus, the biosynthetic pathway of citicoline provides precursors for the synthesis of ACh and phospholipid membranes, including PtdCho, phosphatidylethanolamine (PtEth), and sphingomyelin. Reduced transport and utilization of choline and a concomitant decrease in an essential structural component of cell membranes, PtdCho, as measured in serum, has been observed in elderly populations as compared to younger cohorts. Membrane phospholipids provide the structural building blocks of cell membranes and also play an important role in signal transduction, ion channel and receptor function, regulation of enzymes, and transcriptional activity. The onset of the age-related decline in choline transport has not been well characterized, with the majority of studies comparing young subjects (40 years and younger) to older subjects (60 years and older). It is plausible that declines in active transport of choline may begin to occur prior to manifestation of memory deficits; however, this hypothesis has not been empirically investigated.

Treatment with citicoline also has been shown to modify mitochondrial and synaptosomal proteins and improve brain metabolism in rats, perhaps related to an increase in the availability of cytidylic nucleotides and content of total adenine nucleotides (Adibhatla and Hatcher, J Neurosci Res 70:133-139, 2002). Thus, citicoline is likely to alter multiple biochemical parameters, in part because of the reciprocal relationship between synthesis and function of phospholipid membranes and efficient energy production and utilization provided by mitochondria. Changes in phospholipid membranes and high-energy phosphates may therefore underlie the therapeutic efficacy of citicoline in reducing age- and Alzheimer-related decrements in cognitive functioning, particularly in frontally-mediated abilities involving memory.

Phosphorus-31 magnetic resonance spectroscopy (31P MRS) provides a means of detecting changes in phosphorus-containing metabolites that are associated with levels of high energy phosphate metabolites and constituents of membrane synthesis, indicating cellular bioenergetic state and integrity and function of cell membranes, respectively. Using this method, Babb and colleagues (Babb, Psychopharmacology (Berl) 161:248-254, 2002) observed a significant increase in phosphodiesters (PDE, phospholipid membrane catabolites) at 1.5 Tesla after 6 weeks of citicoline treatment in elderly subjects (69.4±5.6 years). Although the citicoline-related alterations were not regionally specific, as ³¹P spectra were acquired from a 5mm axial brain slice prescribed through the frontal and occipital cortices, the findings were consistent with previous cell culture data, which document increased phospholipid synthesis and turnover. The increase in phospholipid catabolites was correlated with improved performance on a test of verbal learning and memory (California Verbal Learning Test, CVLT).

### Cytidine-Containing Compounds

Cytidine-containing compounds useful in the present invention include any compound including one of the following: cytidine, CMP, CDP, CTP, dCMP, dCDP, and dCTP. Cytidine-containing compounds can also include analogs of cytidine. A preferred cytidine-containing compound is CDP-choline, frequently prepared as a sodium salt. Cytidine-containing compounds, e.g., cytidine and CDP-choline, are commercially available, e.g., from Sigma Chemical Company (St. Louis, Mo.).

CDP-choline is a naturally occurring compound that is hydrolyzed into its components of cytidine and choline in vivo. CDP-choline is synthesized from cytidine-5′-triphosphate and phosphocholine with accompanying production of inorganic pyrophosphate in a reversible reaction catalyzed by the enzyme CTP:phosphocholine cytidyltransferase (CT) (Weiss, Life Sciences 56:637-660, 1995).

### Uridine-Containing Compounds

Uridine and uridine-containing compounds also provide useful therapies because these compounds can be converted to CTP (Wurtman et al., Biochemical Pharmacology 60:989-992, 2000). Useful uridine-containing compounds include, without limitation, any compound comprising uridine, UTP, UDP, or UMP. A preferred uridine-containing compound is triacetyl uridine (2′,3′,5′-tri-O-acetyluridine). Uridine and uridine-containing compounds and analogs are well tolerated in humans.

Formulations and Combination with Other Compounds

The methods and compositions of the invention may employ a cytidine-containing or uridine-containing compound formulated in a nutraceutical composition, e.g., a nutraccutical drink. A composition may optionally contain, in addition to a cytidine- or uridine-containing compound, other FDA-approved food additives and nutriments suitable for human consumption. In other embodiments, the nutraceutical composition additionally contains B vitamins, e.g., vitamin B6, vitamin B12, niacin, and folic acid; amino acids, e.g., tyrosine and phenylalanine; taurine; malic acid; glucuronolactone; and caffeine. Other additives may include, without limitation, phospholipid precursors, e.g., inositol, inositol derivatives, oleic acid, linoleic acid, erucic acid, arachidic acid, stearic acid, palmitic acid, arachidonic acid, alpha-linolenic acid, eicosapentaenoic acid, docosahexaenoic acid, choline, sphingosine, and glycerol; sugars, e.g., sucrose, isomerized sugars, glucose, fructose, trehalose, lactose, xylose, and trehalulose; artificial sweeteners; sugar alcohols, e.g., sorbitol, xylitol, erythritol, lactitol, hydrogenated palatinose, hydrogenated glucose syrup, and reduced malt sugar syrup; emulsifiers, e.g., sucrose fatty acid esters and glycerin; thickeners or stabilizers, e.g., agar-agar, gelatin, carrageenan, guar gum, xanthan gum, pectins and locust bean; sour agents; and fruit juice. A drink may be provided in a relatively small volume, e.g., 20 to 200 milliliters.

Alternatively, the cytidine-containing or uridine-containing compound may be provided in a tablet or capsule. The excipients contained in tablets or capsules may be talc, magnesium stearate, colloidal silicon dioxide, hydrogenated castor oil, sodium carboxy-methylcellulose, or microcrystalline cellulose, or other excipients known in the art. Tablets or capsules may additionally contain any of the same nutriments and additives that may be included in a nutraceutical drink formulation.

For other formulations, conventional pharmaceutical practice may be employed. Methods well known in the art for making formulations are described, e.g., in “Remington: The Science and Practice of Pharmacy” (21st ed.) ed. A. R. Gennaro, 2005, Lippincott, Philadelphia, Pa. For example, other formulations may take the form of a cytidine-containing or uridine-containing compound combined with a pharmaceutically-acceptable diluent, carrier, stabilizer, or excipient. If desired, slow release or extended release delivery systems may be utilized. Biocompatible, biodegradable lactide polymer, lactide/glycolide copolymer, or polyoxyethylene-polyoxypropylene copolymers may be used to control the release of the compounds.

A formulation may contain, e.g., 522.5 mg CDP-choline sodium salt, equivalent to 500 mg of CDP-choline, 261.25 mg CDP-choline sodium salt, equivalent to 250 mg of CDP-choline, or 104.5 mg CDP-choline sodium salt, equivalent to 100 mg of CDP-choline.

### Administration

The preferred route for administration is oral. Oral administration may occur by consumption of a nutraceutical drink, food, tablet, or capsule. Chronic administration is preferred, but occasional administration may also be employed in the methods and compositions of the invention.

Although oral administration is preferred, any other appropriate route of administration may be employed, e.g., parenteral, intravenous, subcutaneous, intramuscular, intracranial, intraorbital, ophthalmic, intraventricular, intracapsular, intraspinal, intracistemal, intraperitoneal, intranasal, or aerosol administration. Formulations for parenteral administration may contain, e.g., excipients, sterile water, saline, polyalkylene glycols such as polyethylene glycol, oils of vegetable origin, or hydrogenated naphthalenes. Other potentially useful parenteral delivery systems include ethylene-vinyl acetate copolymer particles, osmotic pumps, implantable infusion systems, and liposomes. Formulations for inhalation may contain excipients, e.g., lactose, or may be aqueous solutions containing, e.g., polyoxyethylene-9-lauryl ether, glycocholate and deoxycholate, or may be oily solutions for administration in the form of nasal drops, or as a gel.

The dosage preferably ranges from 50 mg per day to 2000 mg per day. The exact dosage of the compound may be dependent, e.g., upon the age and weight of the subject and the route of administration. For example, a human subject of average adult size may orally self-administer CDP-choline at a dosage of 500, 250, or 100 mg daily.

In one particular example, a healthy adult may self-administer a drink containing 250 mg of CDP-choline once daily for a period or six weeks or longer, e.g., greater than one year.

### Example 1

³¹P data were collected using a three-dimensional chemical shift imaging (3D-CSI) technique at 4 Tesla in healthy middle-aged individuals. The use of 3D-CSI at high field has several advantages over previous methods: (1) increased spectral dispersion, which allows for increased precision of metabolite quantification; (2) post-processing grid shifting that allows for 3D placement of voxels of interest; and (3) the ability to co-register voxel placement between baseline and post-treatment follow-up. High-energy metabolite peaks quantified in the present study include phosphocreatine (PCr), β-nucleoside triphosphate (NTP, ATP in brain), and inorganic phosphate (Pi). Phospholipid membrane metabolite peaks quantified include anabolites, phosphomonoesters (PME), including phosphoethanolamine (PE) and phosphocholine (PC), and catabolites, phosphodiesters (PDE), including glycerophosphoethanolamine (GPE) and glycerolphosphocholine (GPC). A region of interest approach was used to examine phosphorus metabolite levels in the anterior cingulate cortex (ACC), given the notable age-related decline in frontal brain metabolism and impairment of frontally-mediated cognitive functions. A comparison region placed in the parieto-occipital cortex (POC) was also evaluated. It was hypothesized that oral supplementation with citicoline would improve frontal bioenergetic metabolism via elevations in availability of high-energy phosphates, as well as alteration of membrane phospholipid turnover by providing additional membrane precursors.

Study Design

Subjects included sixteen neurologically and psychiatrically healthy adults (mean age=47.3±5.4 years; 8 females, 8 males). Trained research technicians administered a structured clinical psychiatric interview using the Structured Clinical Interview for DSM-IV (SCID). All subjects were free of Axis I diagnoses, neurological illness, severe medical problems, and psychoactive substance use. Baseline demographic characteristics of the study sample, including age, education, and handedness, are presented in Table 1.

TABLE 1
Subject Demographics

| | 500 mg Dose | | 2000 mg Dose | |
|---|---|---|---|---|
| | Female | Male | Female | Male |
| Age | 50.3 ± 7.3 | 46.5 ± 5.3 | 45.8 ± 3.1 | 46.5 ± 6.3 |
| Education | 16.8 ± 1.9 | 16.0 ± 2.8 | 14.8 ± 1.9 | 16.3 ± 2.6 |
| Handedness | 3R, 1L | 4R, 0L | 4R, 0L | 2R, 2L |

Data represent mean years ± SD.
“R” = right-handedness,
“L” = left-handedness.

Subjects were randomly assigned to receive a six-week supply of either 500 mg (4 males and 4 females) or 2000 mg (4 males and 4 females) of Cognizin® Citicoline (Kyowa Hakko Kogyo Co., Ltd., JAPAN). Subjects were instructed to take one capsule every day (500 mg group) or four capsules every day in a single dose (2000 mg group) for six weeks. The two doses were selected as previous investigations of elderly, healthy volunteers have demonstrated cognitive enhancement and/or alterations in phosphorus metabolites, as well as minimal side effects. Magnetic resonance spectroscopy (MRS) was completed on all subjects in two imaging sessions: one prior to beginning treatment (baseline) and one after completing six weeks of treatment.

Proton MRI/Phosphorous Magnetic Resonance Spectroscopic Imaging

All proton imaging was performed using the proton channel of the dual tuned open face proton-phosphorus TEM whole-head coil from Bioengineering Inc. (Minneapolis, Minn.) operating at a nominal frequency of 170.3 MHz. A 2D gradient-recalled echo imaging sequence (12 seconds duration) was used to acquire a single image in all three spatial dimensions (sagittal, coronal, axial) to quickly determine the patient's position and angle. High-contrast, T1-weighted sagittal, coronal images as well as T1 and T2-weighted transverse images of the entire brain were acquired using a three-dimensional, magnetization-prepared FLASH imaging sequence (3D-MP FLASH), allowing for clear segmentation between grey-matter, white-matter, and CSF, as well as clearly delineating between the different anatomical regions of interest. This approach was used to optimize ³¹P-MRSI voxel positioning and volumetric correction in the regions of interest.

Phosphorus MRSI was performed using the phosphorus channel of the dual tuned proton-phosphorus TEM whole-head coil from Bioengineering Inc. (Minneapolis, Minn.) operating at 68.9 MHz. Phosphorus MRSI data were recorded using a three-dimensional chemical shift imaging (3D-CSI) sequence (Garraux, Neuroimage 10:149-162, 1999). Acquisition parameters were: TR=500 ms; tip-angle=32 degrees; Rx bandwidth=±2 kHz; complex-points=1024; readout duration=256 ms; pre-pulses=10; pre-acquisition delay=1.905 ms; field of view (FOV) (x,y,z)=330 mm; nominal volume=8.8 cc; maximum phase-encode matrix dimension (x,y,z) 14×14×14 (zero-filled out to 16×16×16 prior to reconstruction). This 3D-CSI sequence employed a reduced phase-encoding scheme based on prior work (Jensen, NMR Biomed 15:338-347, 2002). This scheme allows for the inclusion of spherically bound, reduced-point, weighted k-space acquisition, providing approximately 35% more signal-to-noise for a given scan time and effective voxel volume over conventional methods. The total exam time was approximately 70 minutes to complete the series of MRI and MRSI scans, including patient positioning and magnetic field homogeneity (shim) adjustments performed for each recording.

Snapshots of the 2D gradient-recalled echo imaging sequence (12 seconds duration) used to determine the patient's position and angle (in all three spatial dimensions (sagittal, coronal, axial) were taken and used for co-registration of subject position across study visits.

Data Processing

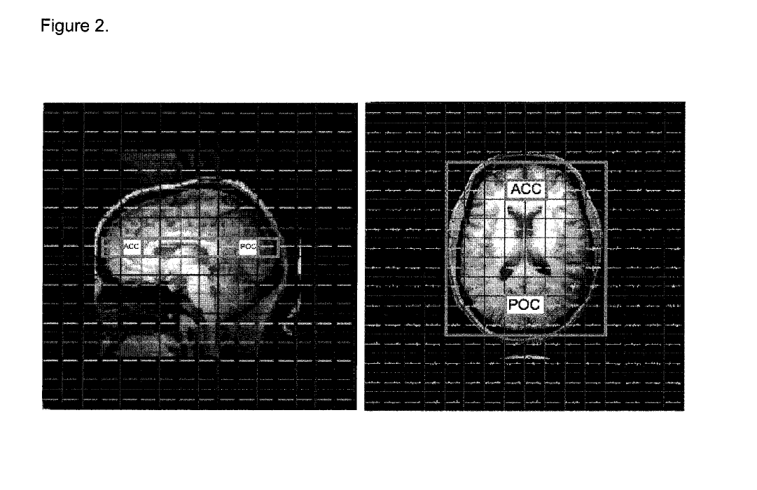

All offline image processing was conducted on a SunBlade100 UNIX workstation (Sun Microsystems, Mountain View, Calif.) using both commercial and custom-written software for the purpose of tissue segmentation and partial-volume analysis. The MRSI grid was shifted in the z dimension, with the center axial image encompassing the genu and splenium of the corpus callosum. The MRSI grid was then shifted in the x and y dimensions so that two 25 cm³ voxels (effective size) were placed in each region of interest, the anterior cingulate cortex (ACC) and a comparison region (parieto-occipital, POC) (FIG. 2). Regions of interest (ROIs) were selected, by a trained rater, with reference to an anatomic atlas (Cranial Neuroimaging and Clinical Neuroanatomy, Kretschmann, H.-J., Thieme Publishing Group, New York, N.Y., 1992), and placements were made on the basis of gyral boundaries and structural landmarks that were visible on the magnetic resonance images (Damasio, Cognition 33:25-62, 1989). Images and coordinates from the post-processing grid shifts used to encompass ACC and comparison regions from data collected at baseline were used to co-register voxel placement across study visits.

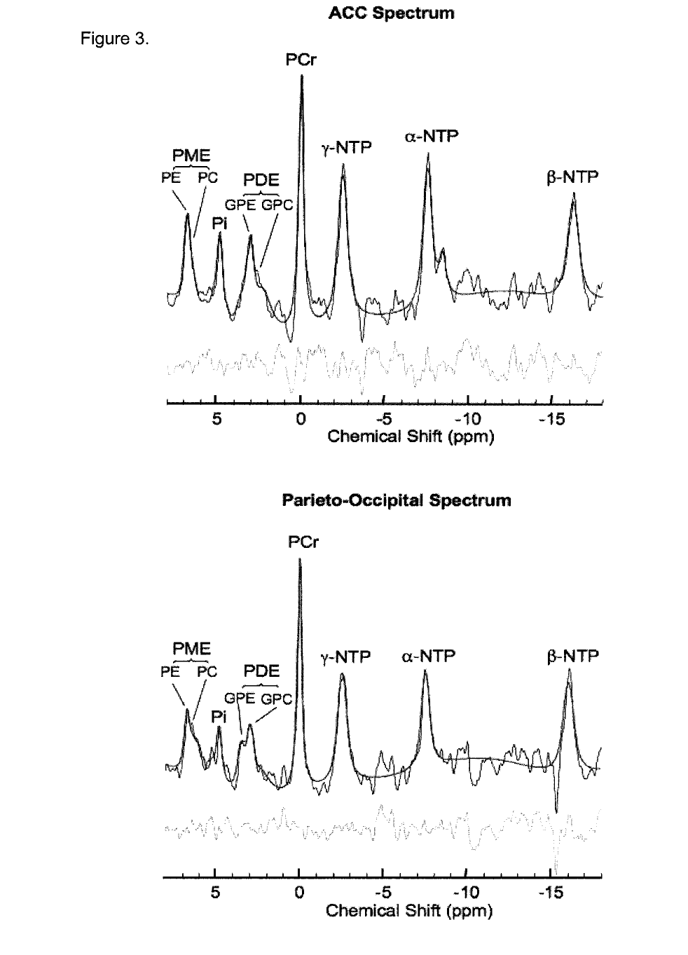

For ³¹P-MRSI spectral analyses, a spectral fitting routine that uses an iterative, non-linear, Marquardt-Levenberg algorithm in combination with prior spectral knowledge was employed to precisely fit the acquired spectra. The phosphorus metabolite peaks quantified included individual metabolites within the PME peak: PE and PC and within the PDE peak: GPE and GPC. Quantification of high-energy phosphorus peaks included Pi, PCr, and β-NTP. The total phosphorus signal (summation of all peaks) was expected to be statistically equivalent between all groups. Thus, each metabolite peak was expressed as percent metabolite, or the ratio of each peak area divided by the sum total of all peak areas at each visit. The ratio of PCr relative to Pi also was examined, given that this ratio has been used to measure energy at steady-state in isolated mitochondria (Gyulai, J Biol Chem 260:3947-3954, 1985) and in dog brain (Nioka, J Appl Physiol 68:2527-2535, 1990) and is thought to reflect phosphorylation potential (Gyulai, supra). A sample of in vivo ³¹P brain spectra from 25 cm³ effective voxels in the ACC and POC of a study subject at 4 Tesla is presented in FIG. 3. Raw data are displayed with modeled fit and residual, with 15 Hz exponential filtering being applied for display.

Data Analysis

Metabolite data were individually analyzed using 2(sex)×2(dose: 500 mg/day or 2000 mg/day)×2(visit: baseline and six weeks) repeated measures analysis of variance (ANOVA). As no significant sex differences, or interactions including sex, were observed, sex was removed as an independent variable in all subsequent analyses. The PCr peak baseline value from one subject (male, high dose) was determined to be an outlier (>3SD) and subsequently was removed from the PCr statistical analysis for both regions. The PC baseline value from one subject (male, high dose) was unable to be fit and was subsequently not included in the PC statistical analysis for the ACC. All data were tested for violations of sphericity, and for post hoc testing, separate repeated measures ANOVAs were conducted to examine the source of visit x dose interactions. SPSS 11.0 (SPSS, Chicago, Ill.) was used for all statistical analyses, with α set at 0.05.

Test-Retest Reliability

Because of a lack of a placebo group in this study, spectroscopic data were collected from an additional 6 healthy subjects (aged 35.7±4.4 years, 3 female) who did not receive citicoline, on two separate visits. Scan 2 was completed 5.9±0.9 weeks after Scan 1. Repeated measures ANOVAs were conducted, similar to those conducted for subjects who received citicoline supplementation.

Results

There were no sex or dose differences at baseline for any of the metabolites examined. Total ³¹P area, which served as the denominator for each metabolite ratio, did not differ significantly between visits, between females and males, or between low and high dose groups, for either the ACC or POC regions. Mean phosphorus metabolite values and total ³¹P area at baseline and at 6 weeks, for the ACC and the POC, are reported in Table 2.

TABLE 2
Metabolite Values at Baseline and Six-Week Follow-up

| ACC | | | POC | | |
|---|---|---|---|---|---|
| Baseline | 6 Weeks | p | Baseline | 6 Weeks | p |

High Energy Phosphate Metabolites

| | | | | | | |
|---|---|---|---|---|---|---|
| PCr | .154 ± .019 | .162 ± .015* | .02 | .164 ± .016 | .173 ± .015 | ns |
| β-NTP | .066 ± .012 | .075 ± .014* | .05 | .063 ± .009 | .066 ± .010 | ns |
| Pi | .073 ± .012 | .065 ± .022 | .15^{A} | .072 ± .010 | .075 ± .013 | ns |
| PCr/Pi | 2.17 ± .46 | 2.82 ± 1.04* | .03^{B} | 2.30 ± .34 | 2.38 ± .53 | ns |

Phospholipid Membrane Anabolites

| | | | | | | |
|---|---|---|---|---|---|---|
| PME | .069 ± .016 | .073 ± .012 | ns | .088 ± .015 | .084 ± .011 | ns |
| PC | .026 ± .010 | .019 ± .008* | .02 | .023 ± .009 | .026 ± .012 | ns |
| PE | .043 ± .015 | .054 ± .008* | .04 | .065 ± .009 | .058 ± .010 | ns |

Phospholipid Membrane Catabolites

| | | | | | | |
|---|---|---|---|---|---|---|
| PDE | .183 ± .032 | .154 ± .049 | .07 | .165 ± .029 | .152 ± .030 | ns |
| GPC | .051 ± .015 | .047 ± .016 | ns | .047 ± .017 | .047 ± .010 | ns |
| GPE | .039 ± .010 | .030 ± .009* | .01 | .034 ± .011 | .032 ± .014 | ns |

Total Phosphorus Signal

| | | | | | |
|---|---|---|---|---|---|
| .366 ± .062 | .337 ± .061 | ns | .284 ± .060 | .295 ± .053 | ns |

*Indicates a significant change from baseline.
Data represent mean (±SD) metabolite ratios relative to the total ³¹P signal at each visit.
^{A}There was a significant visit x dose interaction (p = .03). Post hoc testing revealed decreased Pi only in the low dose group: baseline: .077 ± .012; six weeks: .057 ± .023.
^{B}There was a significant visit x dose interaction (p = .04). Post hoc testing revealed increased PCr/Pi only in the low dose group: baseline: 1.94 ± .22; six weeks: 3.22 ± 1.22.
“ns” indicates difference between visits was not significant (p > .05).

After six-weeks of citicoline administration, regardless of dose, significant increases were seen in the ACC for levels of β-NTP (F(1,14)=4.85,p=0.045), primarily reflecting levels of ATP, and PCr (F(1,13)=6.54,p=0.024), reflecting the high-energy phosphate buffer stores. There was a significant visit x dose interaction for change in levels of ACC Pi after six weeks of treatment (F(1,14)=6.00, p=0.03). Post hoc analyses revealed that Pi levels were significantly reduced in the ACC of subjects who received the 500 mg dose (F(1,7)=9.68, p=0.02; baseline: 0.077±0.012; six weeks: 0.057±0.023), but not in subjects who received the 2000 mg dose (p=0.57; baseline: 0.069±0.010; six weeks: 0.073±0.019). No changes in Pi were observed in the POC at either dose. In addition, there was a significant effect of visit (F(1,13)=6.34, p=0.03) and a significant visit x dose interaction (F(1,13)=4.98,p=0.04) for the PCr/Pi ratio in the ACC. Post hoc analyses revealed that the PCr/Pi ratio in the ACC increased significantly in subjects who received the 500 mg dose (F(1,7)=10.64,p=0.01; baseline: 1.94±0.22; six weeks: 3.22±1.22), but not in subjects who received the 2000 mg dose (p=0.84; baseline: 2.41±0.52; six weeks: 2.33±0.61). No changes in the PCr/Pi ratio were observed in the POC at either dose.

Significant alterations in membrane phospholipids also were observed between baseline and six weeks. Although overall levels of phospholipid precursors (PME) did not differ significantly from baseline in the ACC (p=0.53), there was a significant decrease in levels of PC (F(1,13)=7.76, p=0.02) and a significant increase in levels of PE (F(1,14)=5.23,p=0.04). A trend for decreased overall phospholipid catabolites (PDE) (p=0.06) was observed in the ACC, as well as a significant decrease in GPE from baseline in the ACC (F(1,14)=8.41,p=0.01). No significant changes in phospholipid membrane metabolites were observed in the POC region of interest.

Effects sizes (f) observed for each of the significant metabolite differences in the ACC were medium to large (Cohen J. Statistical power analysis for the behavioral sciences (2nd edition). Erlbaum: Hillsdale, N.J., 1988): ↑β-NTP=0.27, ↑PCr=0.30, ↓Pi=0.38, and ↑PCr/Pi=0.39; ↑PE=0.28, ↓PC=0.32, and ↓GPE=0.32; ↓PDE (trend)=0.26.

Test-Retest Group

Mean phosphorus metabolite values and total ³¹P area at scan 1 and scan 2 in test-retest subjects, for the ACC and the POC, are reported in Table 3. Repeated measures ANOVAs revealed no significant effects of visit (p>0.05) for any of the metabolites examined, either in the ACC or the POC, in the test-retest subjects who did not receive citicoline. These findings indicate that the spectroscopic measurements were consistent over a six-week period, which minimizes the likelihood that the observed metabolite changes following citicoline administration were the sole result of chance or scanner drift.

The results of this study are the first to demonstrate regionally specific changes in high-energy phosphate and membrane phospholipid metabolites after six weeks of citicoline supplementation in healthy middle-aged individuals. The significant citicoline-related metabolite alterations were observed only in the ACC region, some of which were dose dependent, and included increased PCr (↑7%), β-NTP (↑14%), PCr/Pi (↑32%; low dose, ↑66%), and PE (↑26%), and decreased Pi (low dose only, ↓26%), PC (↓29%), and GPE (↓23%). These neurochemical alterations reflect an improvement in brain bioenergetic metabolism and synthesis and turn over of phospholipid membranes.

The results of this study are the first to demonstrate regionally specific changes in high-energy phosphate and membrane phospholipid metabolites after six weeks of citicoline supplementation in healthy middle-aged individuals. The significant citicoline-related metabolite alterations were observed only in the ACC region, some of which were dose dependent, and included increased PCr (↑7%), β-NTP (↑14%), PCr/Pi (↑32%; low dose, ↑66%), and PE (↑26%), and decreased Pi (low dose only, ↓26%), PC (↓29%), and GPE (↓23%). These neurochemical alterations reflect an improvement in brain bioenergetic metabolism and synthesis and turn over of phospholipid membranes.

TABLE 3
Metabolite Values in Test-Retest Subjects

| ACC | | | POC | | |
|---|---|---|---|---|---|
| Scan 1 | Scan 2 | p | Scan 1 | Scan 2 | p |

High Energy Phosphate Metabolites

| | | | | | | |
|---|---|---|---|---|---|---|
| PCr | .156 ± .028 | .154 ± .026 | ns | .168 ± .036 | .165 ± .017 | ns |
| β- | .070 ± .011 | .078 ± .011 | ns | .066 ± .017 | .065 ± .015 | ns |
| NTP | | | | | | |
| Pi | .084 ± .012 | .076 ± .010 | ns | .069 ± .015 | .074 ± .024 | ns |
| PCr/ | 1.89 ± .50 | 1.95 ± .16 | ns | 2.07 ± .55 | 1.97 ± .76 | ns |
| Pi | | | | | | |

Phospholipid Membrane Anabolites

| | | | | | | |
|---|---|---|---|---|---|---|
| PME | .085 ± .017 | .081 ± .019 | ns | .084 ± .019 | .084 ± .018 | ns |
| PC | .030 ± .016 | .024 ± .022 | ns | .025 ± .018 | .027 ± .013 | ns |
| PE | .055 ± .020 | .057 ± .018 | ns | .059 ± .014 | .057 ± .012 | ns |

Phospholipid Membrane Catabolites

| | | | | | | |
|---|---|---|---|---|---|---|
| PDE | .170 ± .068 | .146 ± .057 | ns | .138 ± .024 | .168 ± .037 | ns |
| GPC | .045 ± .028 | .058 ± .013 | ns | .055 ± .005 | .051 ± .015 | ns |
| GPE | .038 ± .011 | .026 ± .020 | ns | .042 ± .028 | .040 ± .024 | ns |

Total Phosphorus Signal

| | | | | | |
|---|---|---|---|---|---|
| .337 ± .072 | .280 ± .072 | ns | .288 ± .062 | .281 ± .037 | ns |

Data represent mean (±SD) metabolite ratios relative to the total ³¹P signal at each visit. No significant differences were observed between scan 1 and scan 2 (5.9 ±0.9 weeks after scan 1).
“ns” indicates difference between visits was not significant (p > .05).

### CONCLUSIONS

Under steady-state conditions, the rate of ATP synthesis equals the rate of ATP utilization, via suppression of excessive glycolysis and activation of mitochondrial oxidative phosphorylation. In the absence of additional glucose, however, ATP levels remain constant since high-energy PCr serves as a buffer for maintenance of ATP levels, when turnover is high or synthesis is low (Bessman, Annu Rev Biochem 54:831-862, 1985), as well as a shuttle for energy from sites of production to sites of utilization (Bessman, Science 211:448-452, 1981; Wallimann, Biochem J 281:21-40, 1992). Availability of PCr pushes the creatine-kinase reaction to generate ATP, via conversion to creatine and high-energy phosphate, at rates that are much faster than oxidative phosphorylation or glycolysis (Wallimann, Biochem J 281:21-40, 1992). This conversion results in a drop in PCr levels while levels of ADP and Pi increase to support steady-state levels of ATP (Gyulai, supra). In this regard, the ratio of PCr relative to Pi has been shown to reflect phosphorylation potential (Nioka, supra). In the present study, citicoline treatment was associated with significant increases in PCr, β-NTP, and the PCr/Pi ratio, as well as decreased Pi (significant at the low dose). The direction of these alterations is consistent with an increase in biocnergetic metabolism, i.e., increased ATP utilization and synthesis (Gyulai, supra; Chance, PNAS 77:7430-7434, 1980; Chance, Ann NY Acad Sci 488:140-153, 1986). This improvement in bioenergetic metabolism following citicoline treatment may be due, in part, to adaptive modifications of mitochondrial proteins that influence electron chain transport, leading to an enhancement of cerebral energy transduction (Villa, Int J Dev Neurosci 11:83-93, 1993). Improved energy availability and utilization may also be directly related to increased synthesis and decreased breakdown of phospholipid membranes (Farooqui, Neurochem Res 29:1961-1977, 2004). Reductions in energy metabolism and mitochondrial abnormalities have been shown to be associated with increased phospholipid breakdown, as measured using MRS, in Alzheimer's populations (Farber, FASEB J 14:2198-2206, 2000). Most notably, regionally specific increases in frontal brain bioenergetic metabolism, and phospholipid maintenance may contribute to the therapeutic effects of citicoline on memory disturbances by increasing vigilance and working memory capacity, but also by reducing mental fatigue (Kato, Neuropsychobiology 39:214-218, 1999). This is consistent with work by Alvarez and colleagues (Alvarez, Methods Find Exp Clin Pharmacol 19:201-210, 1997), who reported that improvements in the memory performance of elderly subjects treated with citicoline were related to facilitation of tissue perfusion and oxygenation, particularly in frontal and temporal regions.

There were also significant alterations in membrane phospholipids observed in the current study. Although no overall change in total phospholipid precursors was evident, individual metabolites of the PME peak (PE and PC) changed significantly. Levels of PE increased after treatment, while PC levels decreased. This opposite pattern of change was not surprising, given that ethanolamine-containing lipids and choline-containing lipids have unique roles in contributing to phospholipid membrane synthesis (Eyster, Advances in Physiology Education 31:5-16, 2007). PE has been shown to be more directly involved in the synthesis of phospholipid membranes, whereas PC contributes more to the synthesis of ACh (Eyster, supra). There also was a trend for a reduction in overall phospholipid catabolites (PDE, p=0.07) and a significant reduction in the GPE resonance within PDE, which further supports a citicoline-related change in phospholipid metabolism. These findings are consistent with previous reports that citicoline inhibits phospholipid degradation (Weiss, supra) and enhances synthesis of membrane phospholipids in rat neural tissue and in whole brain (Kennedy, J Biol Chem 222:193-214,1956; Agut, Ann NY Acad Sci 695:318-320, 1993). The current study results differed from those of Babb and colleagues (Babb, supra), although data may not be comparable between studies because of differences in the 31P MRS methods used and subject population studied. In the Babb study, ³¹P MRS data were acquired from a large slab of brain tissue using a lower field strength scanner (1.5 T) from a population of elderly subjects. In the current study, alterations in the building blocks and break down products of phospholipid membranes were found to be regionally-specific, as citicoline-related changes were observed in the ACC but not in the parieto-occipital cortex.

There are limitations to this study that merit discussion. First, a placebo control group was not included in the study design, but rather, each subject served as his or her own control group, being examined at baseline prior to supplementation and again after completion of six weeks of supplementation. Test-retest reliability data collected from an additional group of healthy subjects, however, demonstrate the stability of the 4 T spectroscopic measurements over a six-week study period. Thus, it is unlikely that the observed metabolite changes associated with citicoline administration were the sole result of chance or scanner drift. Additional methodological approaches were taken to reduce variability across study visits, including the use of subject placement in the magnet at baseline to guide placement at the follow-up visit and coregistration of regional spectral extractions across study visits using the post-processing grid shifting capabilities of CSI. There were no significant differences in the total phosphorus signal (summation of all peaks) in either citicoline or test-retest subjects, by region or at either visit. Thus, metabolite values were not confounded by significant differences in the denominator (total phosphorous signal) used to determine metabolite ratios.

A second limitation of this study was the modest sample size. Several significant citicoline-related changes in phosphorus metabolite values were detected, despite a limited number of subjects examined. Furthermore, effect sizes obtained for each of the metabolites that demonstrated significant citicoline-related alterations at the conclusion of the administration period were in the medium to large range (“Statistical power analysis for the behavioral sciences,” (2^{nd} ed.), Cohen, J., Erlbaum, Mahwah, N.J., 1988). Although there was limited power to detect sex or dose effects on phosphorus metabolite values, it is noteworthy that changes in ACC phosphorus metabolites tended to be of greater magnitude in subjects who received the low dose (500 mg) as compared to those receiving the high dose (2000 mg) (see Table 2). Pi levels were significantly reduced, and PCr/Pi significantly elevated in the ACC at six-week follow-up in subjects who received the 500 mg dose, but not in subjects who received the 2000 mg dose. Precursors of endogenous CDP-choline have been shown to increase following exogenous CDP-choline administration. Subsequently, intracellular CDP-choline and PtdCho are synthesized via substrate-dependent activation of unsaturated CTP: phosphocholine cytidylyltransferase, the rate-limiting enzyme necessary for the production of endogenous CDP-choline, and choline phosphotransferase enzymes (Lopez-Coviella, J Neurochem 65:889-894, 1995). CTP-phosphocholine cytidyl transferase (CT) reaches a stable level of expression and activity during the initial phase of exogenous CDP-choline administration, followed by enhancement of PtdCho synthesis and activation of CT during prolonged exposure (Gimenez, Neurosci Lett 273:163-166, 1999). Thus, the low dose may have had a greater influence on phosphorus metabolites than the higher dose because of a regulatory feedback mechanism that could have inhibited alteration of metabolite levels. This mechanism is consistent with the theory that cellular control systems, which include feedback and feed-forward loops, serve to regulate biological networks (Csajka, J Pharmacokinet Pharmacodyn 33:227-279, 2006; Sauro, Conf Proc IEEE Eng Med Biol Soc 1:44-50, 2006).

Contributions of tissue content on metabolite changes associated with citicoline supplementation were not examined in this study. It is likely that the large voxels (25 cm³) selected contained both gray and white matter. To this end, previous studies have used linear regression analysis and found higher concentrations of PCr in gray matter and lower concentrations of β-NTP in white matter, in healthy human subjects (Hetherington, Magn Reson Med 45:46-52, 2001; Mason, Magn Reson Med 39:346-353, 1998). In addition, our interpretation of the observed PME resonance changes should be considered speculative, due in part to these metabolites including other prominent phospholipids, e.g., phosphatidylserine (PtdSer), ethanolamine plasmogen, phosphocholine plasmogen, sphingomyelin, and rigidly-bound phosphodiesters, that cannot be readily quantified in vivo (Cerdan, Magn Resoh Med 3:432-439, 1986; Kwee, Magn Reson Med 6:296-299, 1988).

Significant neurometabolic and neurophysiological alterations, i.e., improved frontal brain bioenergetic metabolism and phospholipid membrane turnover, were observed in healthy adults who receive citicoline supplementation for 6 weeks. Furthermore, citicoline-related alterations in brain neurochemistry were regionally specific, targeting a frontal brain region (ACC) that is implicated in a variety of cognitive functions, including attention, error detection, and memory.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modification and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

What is claimed is:

1. A method of augmenting bioenergetic metabolism in the frontal brain of a neuropsychologically healthy human, said method comprising administering to said human an effective amount of a cytidine-containing or uridine-containing compound.

2. The method of claim 1, wherein said method increases the rate of metabolism in said frontal brain; increases the level of phosphocreatine in said frontal brain; increases the level of β-nucleoside triphosphates in said frontal brain; or increases the ratio of phosphocreatine to inorganic phosphate in said frontal brain.

3. The method of claim 1, wherein said compound is formulated in a nutraceutical composition and is administered orally.

4. The method of claim 1, wherein said human is a child.

5. The method of claim 1, wherein said human is an adult between 21 and 60 years of age.

6. The method of claim 1, wherein said administering is chronic.

7. The method of claim 1, wherein said cytidine-containing compound is CDP.

8. The method of claim 1, wherein said cytidine-containing compound is CDP-choline.

9. The method of claim 8, wherein said effective amount is equal to or less than 500 mg, 250 mg, or 100 mg.

10. The method of claim 3, wherein said nutraceutical composition is a drink, tablet, or capsule.

11. The method of claim 3, wherein said compound is CDP-choline and said nutraceutical composition further comprises one or more of the group consisting of vitamin B6, vitamin B 12, niacin, folic acid, tyrosine, phenylalanine, taurine, malic acid, glucuronolactone, and caffeine.

12. The method of claim 3, wherein said nutraceutical composition further comprises phospholipid precursors.

13. A nutraceutical composition comprising a cytidine-containing or uridine-containing compound in an amount effective to augment the bioenergetic metabolism in the frontal brain of a neuropsychologically healthy human.

14. The composition of claim 13, wherein said cytidine-containing compound is CDP.

15. The composition of claim 13, wherein said cytidine-containing compound is CDP-choline.

16. The composition of claim 15, wherein said effective amount is equal to or less than 500 mg, 250 mg, or 100 mg.

17. The composition of claim 13, wherein said composition is a drink, tablet, or capsule.

18. The composition of claim 13, wherein said composition further comprises one or more of the group consisting of vitamin B6, vitamin B12, niacin, folic acid, tyrosine, phenylalanine, taurine, malic acid, glucuronolactone, and caffeine.

19. The composition of claim 13, wherein said composition further comprises phospholipid precursors.
